SEQUENCE LISTING (21 sequences)

SEQ ID NO: 1 - DNA, 173 bp, synthetic construct
  source 1..173: mol_type = other DNA; note = circRNA expression frame
tgaaaacacgggttattcccctcctggcaggtatataggagccctatcaaagtcgagctaacggaatggggttttctttccctcctcttcaggaattcgtaagacttgcttttgtcagtggggtggctcccaggatgcagggtcccataggaggggaataacccgtgttttca

SEQ ID NO: 2 - DNA, 47 bp, synthetic construct
  source 1..47: PCR_primers = fwd_name: Frame_F, fwd_seq: taaacttaagcttggtaccgagctctgtgaaaacacgggttattccc, rev_name: Frame_R, rev_seq: agcgggtttaaacgggccctctagatgaaaacacgggttattc; mol_type = other DNA
taaacttaagcttggtaccgagctctgtgaaaacacgggttattccc

SEQ ID NO: 3 - DNA, 43 bp, synthetic construct
  source 1..43: PCR_primers = fwd_name: Frame_F, fwd_seq: taaacttaagcttggtaccgagctctgtgaaaacacgggttattccc, rev_name: Frame_R, rev_seq: agcgggtttaaacgggccctctagatgaaaacacgggttattc; mol_type = other DNA
agcgggtttaaacgggccctctagatgaaaacacgggttattc

SEQ ID NO: 4 - DNA, 39 bp, synthetic construct
  source 1..39: PCR_primers = fwd_name: circTNFAIP3_F, fwd_seq: ttccctcctcttcaggccttgtagagcaccatggctgag, rev_name: circTNFAIP3_R, rev_seq: aaaagcaagtcttaccattcgttttcagtgccacaagctt; mol_type = other DNA
ttccctcctcttcaggccttgtagagcaccatggctgag

SEQ ID NO: 5 - DNA, 40 bp, synthetic construct
  source 1..40: PCR_primers = fwd_name: circTNFAIP3_F, fwd_seq: ttccctcctcttcaggccttgtagagcaccatggctgag, rev_name: circTNFAIP3_R, rev_seq: aaaagcaagtcttaccattcgttttcagtgccacaagctt; mol_type = other DNA
aaaagcaagtcttaccattcgttttcagtgccacaagctt

SEQ ID NO: 6 - DNA, 25 bp, synthetic construct
  source 1..25: PCR_primers = fwd_name: q_circTNFAIP3_F, fwd_seq: tcatccacaaagctctcatcgacag, rev_name: q_circTNFAIP3_R, rev_seq: tgctcagccatggtgctctacaag; mol_type = other DNA
tcatccacaaagctctcatcgacag

SEQ ID NO: 7 - DNA, 24 bp, synthetic construct
  source 1..24: PCR_primers = fwd_name: q_circTNFAIP3_F, fwd_seq: tcatccacaaagctctcatcgacag, rev_name: q_circTNFAIP3_R, rev_seq: tgctcagccatggtgctctacaag; mol_type = other DNA
tgctcagccatggtgctctacaag

SEQ ID NO: 8 - DNA, 20 bp, synthetic construct
  source 1..20: PCR_primers = fwd_name: q_GAPDH_F, fwd_seq: tggtgaaggtcggagtgaac, rev_name: q_GAPDH_R, rev_seq: ggaagatggtgatgggatttc; mol_type = other DNA
tggtgaaggtcggagtgaac

SEQ ID NO: 9 - DNA, 21 bp, synthetic construct
  source 1..21: PCR_primers = fwd_name: q_GAPDH_F, fwd_seq: tggtgaaggtcggagtgaac, rev_name: q_GAPDH_R, rev_seq: ggaagatggtgatgggatttc; mol_type = other DNA
ggaagatggtgatgggatttc

SEQ ID NO: 10 - DNA, 37 bp, synthetic construct
  source 1..37: PCR_primers = fwd_name: circHIPK3_F, fwd_seq: ttccctcctcttcaggtatggcctcacaagtcttggt, rev_name: circHIPK3_R, rev_seq: aaaagcaagtcttacctgtagtaccgagattgtagatatgttg; mol_type = other DNA
ttccctcctcttcaggtatggcctcacaagtcttggt

SEQ ID NO: 11 - DNA, 43 bp, synthetic construct
  source 1..43: PCR_primers = fwd_name: circHIPK3_F, fwd_seq: ttccctcctcttcaggtatggcctcacaagtcttggt, rev_name: circHIPK3_R, rev_seq: aaaagcaagtcttacctgtagtaccgagattgtagatatgttg; mol_type = other DNA
aaaagcaagtcttacctgtagtaccgagattgtagatatgttg

SEQ ID NO: 12 - DNA, 20 bp, synthetic construct
  source 1..20: PCR_primers = fwd_name: q_circHIPK3_F, fwd_seq: tggagactgggggaagatga, rev_name: q_circHIPK3_R, rev_seq: cacactaactggctgagggg; mol_type = other DNA
tggagactgggggaagatga

SEQ ID NO: 13 - DNA, 20 bp, synthetic construct
  source 1..20: PCR_primers = fwd_name: q_circHIPK3_F, fwd_seq: tggagactgggggaagatga, rev_name: q_circHIPK3_R, rev_seq: cacactaactggctgagggg; mol_type = other DNA
cacactaactggctgagggg

SEQ ID NO: 14 - DNA, 44 bp, synthetic construct
  source 1..44: PCR_primers = fwd_name: circFluc_F, fwd_seq: ttccctcctcttcagatggaagacgccaaaaacataaagaaagg, rev_name: circFluc_R, rev_seq: aaaagcaagtcttacttacacggcgatctttccgccc; mol_type = other DNA
ttccctcctcttcagatggaagacgccaaaaacataaagaaagg

SEQ ID NO: 15 - DNA, 37 bp, synthetic construct
  source 1..37: PCR_primers = fwd_name: circFluc_F, fwd_seq: ttccctcctcttcagatggaagacgccaaaaacataaagaaagg, rev_name: circFluc_R, rev_seq: aaaagcaagtcttacttacacggcgatctttccgccc; mol_type = other DNA
aaaagcaagtcttacttacacggcgatctttccgccc

SEQ ID NO: 16 - DNA, 24 bp, synthetic construct
  source 1..24: PCR_primers = fwd_name: q_circFluc_F, fwd_seq: ggaaaactcgacgcaagaaaaatc, rev_name: q_circFluc_R, rev_seq: atagccttatgcagttgctctccag; mol_type = other DNA
ggaaaactcgacgcaagaaaaatc

SEQ ID NO: 17 - DNA, 25 bp, synthetic construct
  source 1..25: PCR_primers = fwd_name: q_circFluc_F, fwd_seq: ggaaaactcgacgcaagaaaaatc, rev_name: q_circFluc_R, rev_seq: atagccttatgcagttgctctccag; mol_type = other DNA
atagccttatgcagttgctctccag

SEQ ID NO: 18 - DNA, 42 bp, synthetic construct
  source 1..42: PCR_primers = fwd_name: circPDCoV_S_F, fwd_seq: ttccctcctcttcagatgcagagagctctattgattatgacc, rev_name: circPDCoV_S_R, rev_seq: aaaagcaagtcttacctaccattccttaaacttaaaggac; mol_type = other DNA
ttccctcctcttcagatgcagagagctctattgattatgacc

SEQ ID NO: 19 - DNA, 40 bp, synthetic construct
  source 1..40: PCR_primers = fwd_name: circPDCoV_S_F, fwd_seq: ttccctcctcttcagatgcagagagctctattgattatgacc, rev_name: circPDCoV_S_R, rev_seq: aaaagcaagtcttacctaccattccttaaacttaaaggac; mol_type = other DNA
aaaagcaagtcttacctaccattccttaaacttaaaggac

SEQ ID NO: 20 - DNA, 23 bp, synthetic construct
  source 1..23: PCR_primers = fwd_name: q_circPDCoV_S_F, fwd_seq: ccgtcctttaagtttaaggaatg, rev_name: q_circPDCoV_S_R, rev_seq: gtttatgtaagaagcgatgtgca; mol_type = other DNA
ccgtcctttaagtttaaggaatg

SEQ ID NO: 21 - DNA, 23 bp, synthetic construct
  source 1..23: PCR_primers = fwd_name: q_circPDCoV_S_F, fwd_seq: ccgtcctttaagtttaaggaatg, rev_name: q_circPDCoV_S_R, rev_seq: gtttatgtaagaagcgatgtgca; mol_type = other DNA
gtttatgtaagaagcgatgtgca

(19) United States
(12) Patent Application Publication
(10) Pub. No.: US 2025/0011796 A1
(43) Pub. Date: Jan. 9, 2025

(54) HIGH-EFFICIENCY EXPRESSION VECTOR OF CIRCULAR RIBONUCLEIC ACID (circRNA) AND USE THEREOF

(71) Applicant: Zhejiang University, Hangzhou City (CN)

(72) Inventors: Jiyong ZHOU, Hangzhou City (CN); Liuyang DU, Hangzhou City (CN); Jinyan GU, Hangzhou City (CN); Yan YAN, Hangzhou City (CN); Yulan JIN, Hangzhou City (CN); Weiren DONG, Hangzhou City (CN)

(73) Assignee: Zhejiang University, Hangzhou City (CN)

(21) Appl. No.: 18/763,564

(22) Filed: Jul. 3, 2024

(30) Foreign Application Priority Data
Jul. 7, 2023 (CN) 202310830730.3

(51) Int. Cl. C12N 15/66; C12Q 1/686

(52) U.S. Cl. CPC C12N 15/66; C12Q 1/686

(57) ABSTRACT

Provided is a high-efficiency expression vector of a circular ribonucleic acid (circRNA). The high-efficiency expression vector is an expression framework with a total length of only 173 bp and having a nucleotide sequence shown in SEQ ID NO: 1, and includes an upstream frame sequence of 92 bp, a downstream frame sequence of 75 bp, and a restriction site EcoR I located between the upstream and downstream frame sequences for inserting a gene to be circularized. In the present disclosure, the high-efficiency expression vector of a circRNA can enable an exogenously-inserted sequence to efficiently express the circRNA, thus providing an efficient expression tool for obtaining the circRNA. On the premise of ensuring efficient circularization of the circRNA, lengths of upstream and downstream flanking sequences required for the circularization have been highly optimized to further reduce the complexity of splicing by-products caused by the flanking sequences.

### CROSS REFERENCE TO RELATED APPLICATION

This patent application claims the benefit and priority of Chinese Patent Application No. 2023108307303, filed with the China National Intellectual Property Administration on Jul. 7, 2023, the disclosure of which is incorporated by reference herein in its entirety as part of the present application.

### REFERENCE TO SEQUENCE LISTING

A computer readable XML file entitled “GWP20231109310_seqlist”, that was created on Jun. 14, 2024, with a file size of about 25,748 bytes, contains the sequence listing for this application, has been filed with this application, and is hereby incorporated by reference in its entirety.

### TECHNICAL FIELD

The present disclosure relates to the technical field of molecular biology, and specifically relates to a high-efficiency expression vector of a circular ribonucleic acid (circRNA) and use thereof.

### BACKGROUND

Circular ribonucleic acid (circRNA) is a type of covalently closed single-stranded circular RNA molecule. It has been proven that the circRNA has a sponge function of the microRNA (miRNA), regulates the occurrence and metastasis of various tumor tissues, regulates transcription, and can be used as a biomarker of tumors. The circRNA is formed by back-splicing of pre-messenger RNA (pre-mRNA). The back-splicing events of an endogenous circRNA are mainly regulated by reverse complementary sequences in flanking introns combined with various RNA-binding proteins (RBPs). Overexpression experiments are usually used to study a gene's function by vector construction.

It can be achieved by directly ligating a gene to be studied into the expression vector. While the exogenous expression of circRNA needs the help of flanking sequences upside and downside the insertion site which may be called circRNA “expression frame”.

Actually, circRNA overexpression methods are still immature, and there are still some defects in their overexpression systems. In particular, there is a contradiction between expression vector and expression frame in length. That is, based on a given eukaryotic plasmid, a larger expression frame means a smaller exogenous insert left. Accordingly, a shorter expression frame sequence of the overexpression vector results in a longer foreign gene sequence that can be inserted. However, a shorter expression frame leads to more difficulty in forming the circular RNA. In view of this, it is a cutting-edge difficulty as well as a key issue in this field to shorten a length of the expression frame sequence as much as possible while still accurately back-splicing a target sequence at a specific site and then efficiently generating the circular RNA.

### SUMMARY

An objective of the present disclosure is to provide an expression frame capable of efficiently expressing a circRNA, to provide an expression vector with a circRNA expression frame mentioned above, to provide a construction method of the circRNA expression frame, to provide use of the circRNA expression frame, and to provide a method for expressing a target gene. In the present disclosure, on the basis of shortening a length of the expression frame, a target sequence is accurately back-spliced at a specific site and the circRNA is efficiently generated. Moreover, on the premise of ensuring efficient circularization of the circRNA, lengths of upstream and downstream flanking sequences required for the circularization have been optimized to reduce the complexity of splicing by-products caused by the flanking sequences.

To achieve the above objective, the present disclosure provides the following technical solutions: the present disclosure provides a circRNA expression frame with a nucleotide sequence shown in SEQ ID NO: 1; where

the circRNA expression frame includes an upstream frame sequence, a restriction site for insertion of a circRNA linear gene, and a downstream frame sequence.

The term “upstream frame sequence” refers to: a nucleic acid sequence in a region from base 1 to base 92 in a framework; and

the term “downstream frame sequence” refers to: a nucleic acid sequence in a region from base 99 to base 173 in the framework.

Preferably, the restriction site for insertion of a circRNA linear gene is EcoR I; and

a sequence of 1 bp to 92 bp is the upstream frame sequence, a sequence of 93 bp to 98 bp is the restriction site EcoR I, and a sequence of 93 bp to 173 bp is the downstream frame sequence in the circRNA expression frame.

The present disclosure further provides an expression vector including the circRNA expression frame.

Preferably, the expression vector is a eukaryotic expression vector.

The present disclosure further provides a construction method of the circRNA expression frame, including the following steps: designing amplification primers to allow PCR amplification on the sequences including upstream frame sequence, the intermediate restriction site EcoR I, and the downstream frame sequence using a synthesized gene as a template, and then adding a homologous arm sequence of 25 bp to two ends of the frame sequence separately, to recombine the entire expression frame into an expression plasmid, where the reserved EcoR I restriction site is used to insert a target circRNA linear sequence to be studied.

Preferably, the amplification primers in the construction method include:

Frame-F-
(SEQ ID NO: 2)
5′-TAAACTTAAGCTTGGTACCGAGCTCTGTGAAAACACGGGTTATTCC
C-3′,
and
Frame-R-
(SEQ ID NO: 3)
5′-AGCGGGTTTAAACGGGCCCTCTAGATGAAAACACGGGTTATTC-3′.

Preferably, a PCR amplification system of the construction method includes: 25 μL of a 2× Max buffer, 1 μL of dNTP, 2 μL of each of upstream and downstream primers (10 mM), 1 μL of a synthesized expression frame DNA template (100 ng), 1 μL of a Phanta® Max Super-Fidelity DNA Polymerase, and sterile water as the balance to 50 μL; and

a reaction program includes: initial denaturation at 95° C. for 5 min; 30 cycles of denaturation at 95° C. for 30 s, annealing at 56° C. for 30 s, and extension at 72° C. for 30 s; extension at 72° C. for 5 min; and storage at 4° C.

In the present disclosure, the circRNA expression frame is used to express a target gene.

The present disclosure further provides a method for expressing a target gene, including: cleaving the circRNA expression frame at the EcoR I endonuclease site, and then recombining a linear sequence of the target gene into the circRNA expression frame to allow expression.

In the present disclosure, the circRNA expression frame is designed according to a splicing mechanism of the circRNA. The expression frame includes an upstream frame sequence, a restriction site EcoR I, and a downstream frame sequence. The expression frame can efficiently circularize a target RNA sequence. The users can directly recombine the linear sequence of a gene that needs to be circularized into a eukaryotic expression vector loaded with the expression frame through the restriction site, and then transfect a resulting recombinant plasmid into cells to express the target circRNA molecule; and

the circRNA expression frame and the expression vector thereof provide a tool for efficient expression of the circRNA.

Compared with the prior art, the present disclosure has the following beneficial effects:

The present disclosure provides a high-efficiency expression vector of a circRNA. The high-efficiency expression vector is an expression framework with a total length of only 173 bp and having a nucleotide sequence shown in SEQ ID NO: 1, and includes an upstream frame sequence of 92 bp, a downstream frame sequence of 75 bp, and a restriction site EcoR I located between the upstream and downstream frame sequences for inserting a gene to be circularized. In the present disclosure, the high-efficiency expression vector of a circRNA can enable an exogenously-inserted sequence to efficiently express the circRNA, thus providing an efficient expression tool for obtaining the circRNA. On the premise of ensuring a high circularization efficiency of the circRNA, lengths of upstream and downstream flanking sequences required for the circularization have been highly optimized to further reduce the complexity of splicing by-products caused by the flanking sequences.

### BRIEF DESCRIPTION OF THE DRAWINGS

The drawings are provided for further understanding of the present disclosure and constitute a part of the specification. The drawings, together with the embodiments of the present disclosure, are intended to explain the present disclosure, rather than to limit the present disclosure. In the accompanying drawings:

FIG. 1 shows a composition of the circRNA expression frame in the present disclosure;

FIG. 2 shows an agarose gel electrophoresis diagram for PCR amplification of the circRNA expression frame in the present disclosure;

FIG. 3 shows a structure of a eukaryotic expression vector of the circRNA expression frame in the present disclosure;

FIG. 4 shows quantitative PCR results of the circRNA overexpression vector in the present disclosure inserted into a circRNA TNFAIP3 gene;

FIG. 5 shows quantitative PCR results of the circRNA overexpression vector in the present disclosure inserted into a circRNA HIPK3 gene;

FIG. 6 shows quantitative PCR results of the circRNA overexpression vector in the present disclosure inserted into a linear gene Fluc; and

FIG. 7 shows quantitative PCR results of the circRNA overexpression vector in the present disclosure inserted into a PDCoV S gene.

### DETAILED DESCRIPTION OF THE EMBODIMENTS

The preferred embodiments of the present disclosure are described below with reference to the drawings. It should be understood that the preferred embodiments described herein are only used to illustrate the present disclosure, rather than to limit the present disclosure.

### Example 1: Design of a circRNA Expression Frame

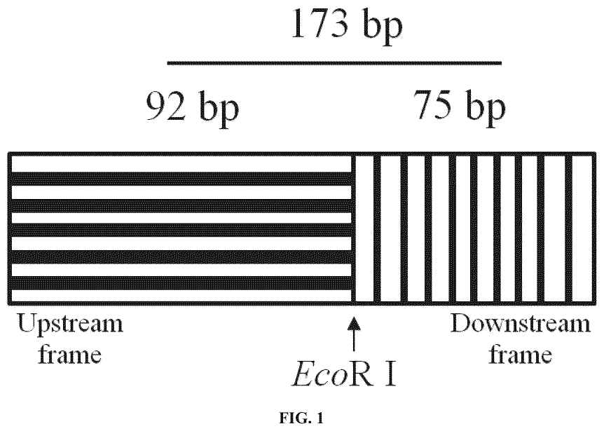

In the present disclosure, a designed circRNA expression frame included an upstream frame sequence, a circRNA insertion restriction site, and a downstream frame sequence. The frame had a base sequence shown in SEQ ID NO: 1, where a sequence of 1 bp to 92 bp was the upstream frame sequence (92 bp); a sequence of 93 bp to 98 bp was the restriction site EcoR I; and a sequence of 99 bp to 173 bp was the downstream frame sequence (75 bp). The frame had a composition shown in FIG. 1.

### Example 2: Obtaining a Frame Sequence of the circRNA Overexpression Frame

A complete frame sequence was chemically synthesized according to the design scheme of the circRNA expression frame in Example 1. A DNA of the synthesized expression frame was used as a template, and upstream and downstream amplification primers of the frame sequence were designed to allow PCR amplification. The reconstructed frame sequence containing 25 bp homologous arms at both ends was ligated to a plasmid vector using homologous recombination.

The present disclosure adopted the following specific technical solutions:

### 1. Design of PCR Amplification Primers:

Frame-F-5′-TAAACTTAAGCTTGGTACCGAGCTCTGTGAAAACACGGGTTATTCCC-3′ (SEQ ID NO: 2),
Frame-R-5′-AGCGGGTTTAAACGGGCCCTCTAGATGAAAACACGGGTTATTC-3′ (SEQ ID NO: 3); and
an amplified fragment was 223 bp.
   2. PCR Amplification of the circRNA Expression Frame:
A 50 μL PCR reaction system was prepared with a high-fidelity enzyme Phanta® Max Super-Fidelity DNA Polymerase (Vazyme) and the above primers, including: 25 μL of a 2× Max buffer, 1 μL of dNTP, 2 μL of each of upstream and downstream primers (10 mM), 1 μL of a synthesized expression frame DNA template (100 ng), 1 μL of a Phanta® Max Super-Fidelity DNA Polymerase, and sterile water as the balance to 50 μL; and
a reaction program included: initial denaturation at 95° C. for 5 min; 30 cycles of denaturation at 95° C. for 30 s, annealing at 56° C. for 30 s, and extension at 72° C. for 30 s; extension at 72° C. for 5 min; and storage at 4° C.

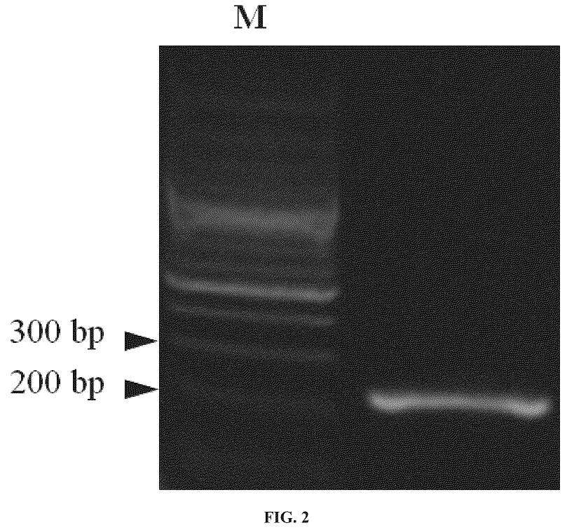

After the PCR amplification was completed, 5 μL of a PCR product was collected to allow 1% agarose gel electrophoresis. The agarose gel electrophoresis results of PCR amplification of the circRNA expression frame were shown in FIG. 2, where M represented a 100 bp DNA ladder, and arrows on the right side represented a target band of the circRNA expression frame of the present disclosure.

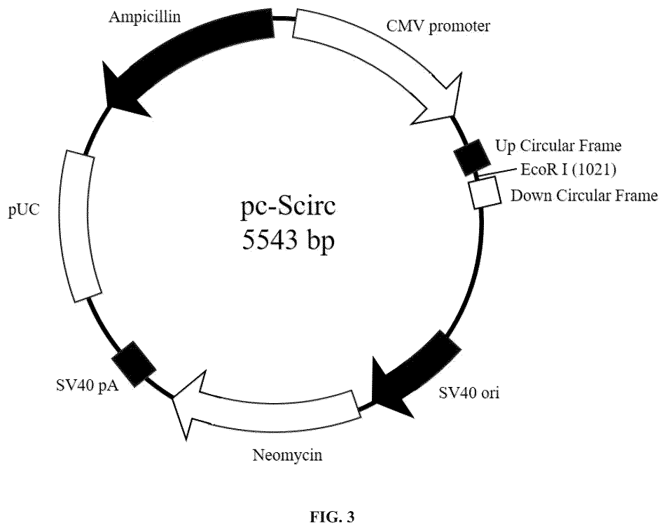

The PCR product was cut and recovered, and purified using a gel recovery kit. A eukaryotic expression vector pcDNA3.1 (+) was double digested with BamH I and Xho I and then purified using a cleaning recovery kit. The recovered frame DNA was recombined into the BamH I/Xho I double-digested pcDNA3.1 (+) vector to obtain a new plasmid pc-Scirc containing a circRNA expression frame, with a model map shown in FIG. 3.

### Example 3: The Overexpression of circRNA TNFAIP3 Based on Pc-Scirc

PCR amplification primers were designed based on the circRNA TNFAIP3 gene to amplify a linear sequence of the circRNA, with a nucleotide sequence length of 310 bp. The target nucleotide sequence was recombined into the circRNA overexpression frame pc-Scirc of the present disclosure through the EcoR I restriction site, so as to construct a pc-Scirc-TNFAIP3 overexpression plasmid. This vector was transfected into HEK-293 cells to detect its expression efficiency.

After detection by quantitative PCR, the constructed circRNA overexpression vector could efficiently express the target circRNA. The detection specifically included the following steps:

1. Design of PCR Amplification Primers for circRNA TNFAIP3:

Forward and reverse primers were designed using Primer Premier 5.0, and a 15 bp homologous arm sequence was added to a 5′-end of the forward primer and the reverse primer separately for homologous recombination with a pcDNA3.1 (+) plasmid. Primer sequences were as follows:

circTNFAIP3-F-
(SEQ ID NO: 4)
5′-TTCCCTCCTCTTCAGGCCTTGTAGAGCACCATGGCTGAG-3′,
circTNFAIP3-R-
(SEQ ID NO: 5)
5′-AAAAGCAAGTCTTACCATTCGTTTTCAGTGCCACAAGCTT-3′.

2. PCR Amplification of circRNA TNFAIP3 Sequence:

A 50 μL PCR reaction system was prepared with a high-fidelity enzyme Phanta® Max Super-Fidelity DNA Polymerase (Vazyme) and the above primers, including: 25 μL of a 2× Max buffer, 1 μL of dNTP, 2 μL of each of upstream and downstream primers (10 mM), 1 μL of a cDNA reverse transcripted of the RNA isolated from ST cells, 1 μL of a Phanta® Max Super-Fidelity DNA Polymerase, and sterile water as the balance; and

a reaction program included: initial denaturation at 95° C. for 5 min; 30 cycles of denaturation at 95° C. for 30 s, annealing at 56° C. for 30 s, and extension at 72° C. for 30 s; extension at 72° C. for 5 min; and storage at 4° C. The PCR product was recovered through gel cutting and then recombined into a pc-Scirc vector through the restriction site EcoR I, and a resulting new plasmid was named pc-Scirc-TNFAIP3.

3. Quantitative PCR Detection of circRNA Expressed by Pc-Scirc-TNFAIP3:

pc-Scirc and pc-Scirc-TNFAIP3 were transfected into HEK-293 cells using a Biobest transfection reagent at a transfection concentration of 1 μg/mL, and real quantitative PCR was conducted to detect the expression of the circRNA 24 h after transfection.

The “back-to-back” primers used to amplify an adapter sequence including the circRNA TNFAIP3 were designed using Primer Premier5.0, as follows:

q-circTNFAIP3-F-
(SEQ ID NO: 6)
5′-TCATCCACAAAGCTCTCATCGACAG-3′,
q-circTNFAIP3-R-
(SEQ ID NO: 7)
5′-TGCTCAGCCATGGTGCTCTACAAG-3′.

A GAPDH gene as a reference gene was used as a correction control for the quantitative results, and primer sequences were as follows:

q-GAPDH-F-
(SEQ ID NO: 8)
5′-TGGTGAAGGTCGGAGTGAAC-3′,
q-GAPDH-R-
(SEQ ID NO: 9)
5′-GGAAGATGGTGATGGGATTTC-3′.

A specific detection method was as follows:

### (1) Extraction of Total Cellular RNA:

The total cellular RNA was extracted from HEK-293 cells transfected with pc-Scirc and pc-Scirc-TNFAIP3 plasmids strictly according to the instructions of an RNA isolator Total RNA Extraction Reagent:

100,000 cells were added into 400 μL of RNA isolator reagent.

200 μL of chloroform was added to the cells, shaken vigorously for 10 s, and allowed to stand at room temperature for 15 min; after centrifugation at 4° C. and 12,000 rpm for 10 min, the liquid was divided into three layers; an uppermost aqueous phase solution was transferred into a new RNase free EP tube, added with an equal volume of isopropyl alcohol, mixed well by placing up and down, and allowed to stand at room temperature for 10 min.

After centrifugation at 12,000 rpm for 10 min at 4° C., a white RNA precipitate appeared at the bottom of the tube; the supernatant was discarded, 1 mL of 75% ethanol was added into the tube, gently inverted several times, and centrifuged at 12,000 rpm for 5 min at 4° C.

The liquid in the tube was discarded, and the white RNA precipitate was air-dried at room temperature until it became transparent, and dissolved in 40 μL of DEPC water.

(2) Reverse Transcription of RNA into cDNA:

The total RNA was reverse transcribed using HiScriptII QRT Super Mix for qPCR (+gDNA wiper). The reaction system and reaction conditions were as follows: the reaction system included 950 ng of RNA, 4 μL of 4×gDNA wiper Mix, and ddH₂O making up to 16 μL; after mixing well, a resulting system was placed in a 42° C. water bath for 2 min, added with 4 μL of 5×HiScript II qRT Super Mix II, mixed evenly to allow reverse transcription at 50° C. for 15 min and then 85° C. for 5 s. The obtained cDNA was stored in a −40° C. refrigerator for subsequent qPCR detection.

(3) Quantitative Detection of circRNA TNFAIP3:

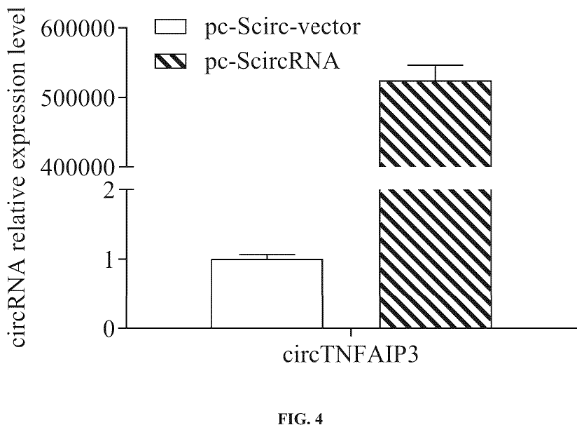

The expression level of circRNA TNFAIP3 in the cDNA obtained in the previous step was detected using the AceQ qPCR SYBR Green Master Mix kit (Vazyme). A detection system included: 10 μL of 2×AceQ qPCR SYBR Green MasterMix, 0.4 μL each of 10 μM upstream and downstream primers, 4 μL of cDNA, and 5.2 μL of ddH₂O. A qPCR program included: initial denaturation at 95° C. for 5 min, 1 cycle; 95° C. for 10 s, 60° C. for 30 s, and 95° C. for 15 s, 40 cycles; and 60° C. for 30 s, 95° C. for 15 s, 1 cycle. The quantification results showed that the expression level of circRNA in HEK-293 cells transfected with pc-Scirc-TNFAIP3 plasmid was more than 500,000 times higher than that in HEK-293 cells transfected with control plasmid pc-Scirc. The quantification results of the circRNA TNFAIP3 gene inserted into the circRNA overexpression vector were shown in FIG. 4.

### Example 4: The Overexpression of circRNA HIPK Based on Pc-Scirc

PCR amplification primers were designed based on the circRNA HIPK3 gene to amplify a linear sequence of the circRNA, with a nucleotide sequence of 1,099 bp.

The target nucleotide sequence was recombined into the circRNA overexpression frame pc-Scirc of the present disclosure through the EcoR I restriction site, so as to construct a pc-Scirc-HIPK3 overexpression plasmid. This vector was transfected into HEK-293 cells to detect its expression efficiency.

After detection by quantitative PCR, the constructed circRNA overexpression frame could efficiently express the target circRNA. The detection specifically included the following steps:

1. Design of PCR Amplification Primers for circRNA HIPK3:

Forward and reverse primers were designed using Primer Premier 5.0, and a 15 bp homologous arm sequence was added to a 5′-end of the forward primer and the reverse primer separately for homologous recombination with a pcDNA3.1 (+) plasmid. Primer sequences were as follows:

circHIPK3-F-
(SEQ ID NO: 10)
5′-TTCCCTCCTCTTCAGGTATGGCCTCACAAGTCTTGGT-3′,
circHIPK3-R-
(SEQ ID NO: 11)
5′-AAAAGCAAGTCTTACCTGTAGTACCGAGATTGTAGATATGTTG-3′.

2. PCR Amplification of circRNA HIPK3 Sequence:

A 50 μL PCR reaction system was prepared with a high-fidelity enzyme Phanta® Max Super-Fidelity DNA Polymerase (Vazyme) and the above primers, including: 25 μL of a 2× Max buffer, 1 μL of dNTP, 2 μL of each of upstream and downstream primers (10 mM), 1 μL of a cDNA reverse transcripted of the RNA isolated from HEK-293 cells, 1 μL of a Phanta® Max Super-Fidelity DNA Polymerase, and sterile water as the balance; and

a reaction program included: initial denaturation at 95° C. for 5 min; 30 cycles of denaturation at 95° C. for 30 s, annealing at 56° C. for 30 s, and extension at 72° C. for 1 min; extension at 72° C. for 5 min; and storage at 4° C.

The PCR product was recovered through gel cutting and then recombined into a pc-Scirc vector through the restriction site EcoR I, and a resulting new plasmid was named pc-Scirc-HIPK3.

3. Quantitative PCR Detection of circRNA Expressed by Pc-Scirc-HIPK3:

pc-Scirc and pc-Scirc-HIPK3 were transfected into HEK-293 cells using a Biobest transfection reagent at a transfection concentration of 1 μg/mL, and real quantitative PCR was conducted to detect the expression of the circRNA 24 h after transfection.

The “back-to-back” primers used to amplify an adapter sequence including the circRNA HIPK3 were designed using Primer Premier5.0, as follows:

q-circHIPK3-F-
(SEQ ID NO: 12)
5′-TGGAGACTGGGGGAAGATGA-3′,
q-circHIPK3-R-
(SEQ ID NO: 13)
5′-CACACTAACTGGCTGAGGGG-3′.

A GAPDH gene as a reference gene was used as a correction control for the fluorescence quantitative results, and primer sequences were as follows:

q-GAPDH-F-
(SEQ ID NO: 8)
5′-TGGTGAAGGTCGGAGTGAAC-3′,
q-GAPDH-R-
(SEQ ID NO: 9)
5′-GGAAGATGGTGATGGGATTTC-3′.

A specific detection method was as follows:

### (1) Extraction of Total Cellular RNA;

The total cellular RNA was extracted from HEK-293 cells transfected with pc-Scirc and pc-Scirc-HIPK3 plasmids strictly according to the instructions of an RNA isolator Total RNA Extraction Reagent:

100,000 cells were added into 400 μL of RNA isolator reagent.

200 μL of chloroform was added, and a resulting mixture was shaken vigorously for 10 s and placed on ice for 15 min.

After centrifugation at 4° C. and 12,000 rpm for 10 min, the liquid was divided into three layers; an uppermost aqueous phase solution was transferred into a new RNase free EP tube, added with an equal volume of isopropyl alcohol, mixed well by placing up and down, and allowed to stand at room temperature for 10 min.

After centrifugation at 12,000 rpm for 10 min at 4° C., a white RNA precipitate appeared at the bottom of the tube; the supernatant was discarded, 1 mL of 75% ethanol was added into the tube, gently inverted several times, and centrifuged at 12,000 rpm for 5 min at 4° C.

The liquid in the tube was discarded, and the white RNA precipitate was air-dried at room temperature until it became transparent, and dissolved in 40 μL of DEPC water.

(2) Reverse Transcription of RNA into cDNA;

The total RNA was reverse transcribed using HiScript II QRT Super Mix for qPCR (+gDNA wiper). The reaction system and reaction conditions were as follows: the reaction system included 950 ng of RNA, 4 μL of 4×gDNA wiper Mix, and ddH₂O making up to 16 μL; after mixing well, a resulting system was placed in a 42° C. water bath for 2 min, added with 4 μL of 5×HiScript II qRT Super Mix II, mixed evenly to allow reverse transcription at 50° C. for 15 min and then 85° C. for 5 s. The obtained cDNA was stored in a −40° C. refrigerator for subsequent qPCR detection.

(3) Quantitative Detection of circRNA HIPK3;

The expression level of circRNA HIPK3 in the cDNA obtained in the previous step was detected using the AceQ qPCR SYBR Green Master Mix kit (Vazyme). A detection system included: 10 μL of 2×AceQ qPCR SYBR Green MasterMix, 0.4 μL each of 10 μM upstream and downstream primers, 4 μL of cDNA, and 5.2 μL of ddH₂O.

A qPCR program included: initial denaturation at 95° C. for 5 min, 1 cycle; 95° C. for 10 s, 60° C. for 30 s, and 95° C. for 15 s, 40 cycles; and 60° C. for 30 s and 95° C. for 15 s, 1 cycle.

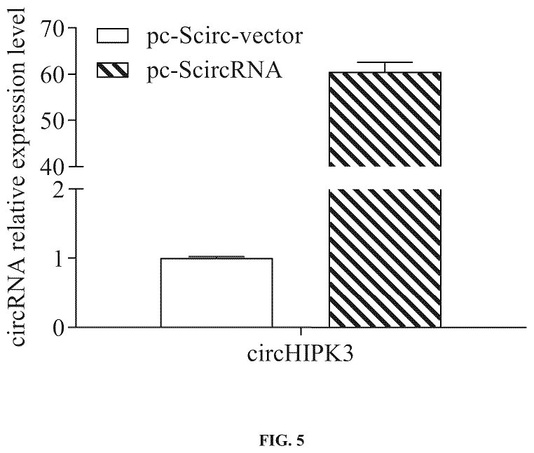

The quantification results showed that the expression level of circRNA in HEK-293 cells transfected with pc-Scirc-HIPK3 plasmid was more than 50 times higher than that in HEK-293 cells transfected with control plasmid pc-Scirc. The quantitation results of the circRNA HIPK3 gene inserted into the circRNA overexpression vector were shown in FIG. 5.

### Example 5: The Overexpression of circRNA Fluc Based on Pc-Scirc

PCR amplification primers were designed based on the firefly luciferase gene (Fluc) gene to amplify a linear sequence of the circRNA, with a nucleotide sequence of 1,653 bp.

The target nucleotide sequence was recombined into the circRNA overexpression frame pc-Scirc of the present disclosure through the EcoR I restriction site, so as to construct a pc-Scirc-Fluc overexpression plasmid. This vector was transfected into HEK-293 cells to detect its expression efficiency.

After detection by quantitative PCR, the constructed circRNA overexpression frame could efficiently express the target circRNA. The detection specifically included the following steps:

### 1. Design of PCR Primers to Amplify a Full Length of the Linear Gene Fluc;

Forward and reverse primers were designed using Primer Premier 5.0, and a 15 bp homologous arm sequence was added to a 5′-end of the forward primer and the reverse primer separately for homologous recombination with a pcDNA3.1 (+) plasmid. Primer sequences were as follows:

circFluc-F-
(SEQ ID NO: 14)
5′-TTCCCTCCTCTTCAGATGGAAGACGCCAAAAACATAAAGAAAG
G-3′,
circFluc-R-
(SEQ ID NO: 15)
5′-AAAAGCAAGTCTTACTTACACGGCGATCTTTCCGCCC-3′.

### 2. PCR Amplification of a Full-Length Sequence of the Linear Gene Fluc;

A 50 μL PCR reaction system was prepared with a high-fidelity enzyme Phanta® Max Super-Fidelity DNA Polymerase (Vazyme) and the above primers, including: 25 μL of a 2× Max buffer, 1 μL of dNTP, 2 μL of each of upstream and downstream primers (10 mM), 1 μL of a pmirGLO plasmid as template (100 ng), 1 μL of a Phanta® Max Super-Fidelity DNA Polymerase, and sterile water as the balance; and

a reaction program included: initial denaturation at 95° C. for 5 min; 30 cycles of denaturation at 95° C. for 30 s, annealing at 56° C. for 30 s, and extension at 72° C. for 2 min; extension at 72° C. for 5 min; and storage at 4° C.

The PCR product was recovered through gel cutting and then recombined into a pc-Scirc vector through the restriction site EcoR I, and a resulting new plasmid was named pc-Scirc-Fluc.

3. Quantitative PCR Detection of circRNA Expressed by Pc-Scirc-Fluc;

pc-Scirc and pc-Scirc-Fluc were transfected into HEK-293 cells using a Biobest transfection reagent at a transfection concentration of 1 μg/mL, and real quantitative PCR was conducted to detect the expression of the circRNA 24 h after transfection.

The “back-to-back” primers used to amplify an adapter sequence including the circRNA Fluc were designed using Primer Premier5.0, as follows:

q-circFluc-F-
(SEQ ID NO: 16)
5′-GGAAAACTCGACGCAAGAAAAATC-3′,
q-circFluc-R-
(SEQ ID NO: 17)
5′-ATAGCCTTATGCAGTTGCTCTCCAG-3′.

A GAPDH gene as a reference gene was used as a correction control for the fluorescence quantitative results, and primer sequences were as follows:

q-GAPDH-F-
(SEQ ID NO: 8)
5′-TGGTGAAGGTCGGAGTGAAC-3′,
q-GAPDH-R-
(SEQ ID NO: 9)
5′-GGAAGATGGTGATGGGATTTC-3′

A specific detection method was as follows:

### (1) Extraction of Total Cellular RNA;

The total cellular RNA was extracted from HEK-293 cells transfected with pc-Scirc and pc-Scirc-Fluc plasmids strictly according to the instructions of an RNA isolator Total RNA Extraction Reagent:

100,000 cells were added into 400 μL of RNA isolator reagent. 200 μL of chloroform was added, and a resulting mixture was shaken vigorously for 10 s and placed on ice for 15 min.

After centrifugation at 4° C. and 12,000 rpm for 10 min, the liquid was divided into three layers; an uppermost aqueous phase solution was transferred into a novel RNase free EP tube, added with an equal volume of isopropyl alcohol, mixed well by placing up and down, and allowed to stand at room temperature for 10 min.

After centrifugation at 12,000 rpm for 10 min at 4° C., a white RNA precipitate appeared at the bottom of the tube; the supernatant was discarded, 1 mL of 75% ethanol was added into the tube, gently inverted several times, and centrifuged at 12,000 rpm for 5 min at 4° C. The liquid in the tube was discarded, and the white RNA precipitate was air-dried at room temperature until it became transparent, and dissolved in 40 μL of DEPC water.

(2) Reverse Transcription of RNA into cDNA;

The total RNA was reverse transcribed using HiScript II QRT Super Mix for qPCR (+gDNA wiper). The reaction system and reaction conditions were as follows: the reaction system included 950 ng of RNA, 4 μL of 4×gDNA wiper Mix, and ddH₂O making up to 16 μL; after mixing well, a resulting system was placed in a 42° C. water bath for 2 min, added with 4 μL of 5×HiScript II qRT Super Mix II, mixed evenly to allow reverse transcription at 50° C. for 15 min and then 85° C. for 5 s. The obtained cDNA was stored in a −40° C. refrigerator for subsequent qPCR detection.

(3) Quantitative Detection of circRNA Fluc;

The expression level of circRNA Fluc in the cDNA obtained in the previous step was detected using the AceQ qPCR SYBR Green Master Mix kit (Vazyme). A detection system included: 10 μL of 2×AceQ qPCR SYBR Green MasterMix, 0.4 μL each of 10 μM upstream and downstream primers, 4 μL of cDNA, and 5.2 μL of ddH₂O.

A reaction program included: initial denaturation at 95° C. for 5 min, 1 cycle;
40 cycles of denaturation at 95° C. for 10 s, annealing at 60° C. for 30 s, and extension at 95° C. for 15 s;
60° C. for 30 s and 95° C. for 15 s, 1 cycle.

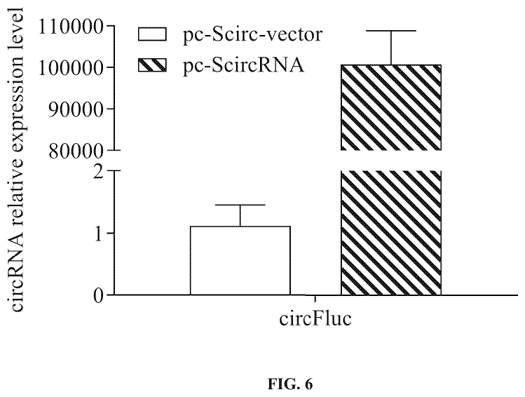

The quantification results showed that the expression level of circRNA in HEK-293 cells transfected with pc-Scirc-Fluc plasmid was 100,000 times higher than that in HEK-293 cells transfected with control plasmid pc-Scirc. The quantitation results of the linear gene Fluc inserted into the circRNA overexpression vector were shown in FIG. 6.

### Example 6: The Overexpression of circRNA of an S Gene of a Porcine Deltacoronavirus (PDCoV) Based on Pc-Scirc

PCR amplification primers were designed based on the S gene of PDCoV to amplify a linear sequence of the circRNA, with a nucleotide sequence of 3,483 bp.

The target nucleotide sequence was recombined into the circRNA overexpression frame pc-Scirc of the present disclosure through the EcoR I restriction site, so as to construct a pc-Scirc-PDCoV-S overexpression plasmid. This vector was transfected into HEK-293 cells to detect its expression efficiency.

After detection by quantitative PCR, the constructed circRNA overexpression frame could efficiently express the target circRNA. The detection specifically included the following steps:

### 1. Design of PCR Primers to Amplify a Full Length of the S Gene of PDCoV;

Forward and reverse primers were designed using Primer Premier 5.0, and a 15 bp homologous arm sequence was added to a 5′-end of the forward primer and the reverse primer separately for homologous recombination with a pcDNA3.1 (+) plasmid. Primer sequences were as follows:

circPDCoV-S-F-
(SEQ ID NO: 18)
5′-TTCCCTCCTCTTCAGATGCAGAGAGCTCTATTGATTATGACC-3′,
circPDCoV-S-R-
(SEQ ID NO: 19)
5′-AAAAGCAAGTCTTACCTACCATTCCTTAAACTTAAAGGAC-3′.

### 2. PCR Amplification of a Full-Length Sequence of the S Gene of PDCoV;

A 50 μL PCR reaction system was prepared with a high-fidelity enzyme Phanta® Max Super-Fidelity DNA Polymerase (Vazyme) and the above primers, including: 25 μL of a 2× Max buffer, 1 μL of dNTP, 2 μL of each of upstream and downstream primers (10 mM), 1 μL of a cDNA reverse transcripted of the RNA isolated from PDCoV, 1 μL of a Phanta® Max Super-Fidelity DNA Polymerase, and sterile water as the balance; and

a reaction program included: initial denaturation at 95° C. for 5 min; 30 cycles of denaturation at 95° C. for 30 s, annealing at 56° C. for 30 s, and extension at 72° C. for 3 min; extension at 72° C. for 5 min; and storage at 4° C.

The PCR product was recovered through gel cutting and then recombined into a pc-Scirc vector through the restriction site EcoR I, and a resulting new plasmid was named pc-Scirc-PDCoV-S.

3. Quantitative PCR Detection of circRNA Expressed by Pc-Scirc-PDCoV-S;

pc-Scirc and pc-Scirc-PDCoV-S were transfected into HEK-293 cells using a Biobest transfection reagent at a transfection concentration of 1 μg/mL, and real quantitative PCR was conducted to detect the expression of the circRNA 24 h after transfection.

The “back-to-back” primers used to amplify an adapter sequence including the circRNA PDCoV-S were designed using Primer Premier5.0, as follows:

q-circPDCoV-S-F-
(SEQ ID NO: 20)
5′-CCGTCCTTTAAGTTTAAGGAATG-3′,
q-circPDCoV-S-R-
(SEQ ID NO: 21)
5′-GTTTATGTAAGAAGCGATGTGCA-3′.

A GAPDH gene as a reference gene was used as a correction control for the fluorescence quantitative results, and primer sequences were as follows:

q-GAPDH-F-
(SEQ ID NO: 8)
5′-TGGTGAAGGTCGGAGTGAAC-3′,
q-GAPDH-R-
(SEQ ID NO: 9)
5′-GGAAGATGGTGATGGGATTTC-3′.

A specific detection method was as follows:

### (1) Extraction of Total Cellular RNA;

The total cellular RNA was extracted from HEK-293 cells transfected with pc-Scirc and pc-Scirc-PDCoV-S plasmids strictly according to the instructions of an RNA isolator Total RNA Extraction Reagent:

100,000 cells were added into 400 μL of RNA isolator reagent.

200 μL of chloroform was added, and a resulting mixture was shaken vigorously for 10 s and placed on ice for 15 min.

After centrifugation at 4° C. and 12,000 rpm for 10 min, the liquid was divided into three layers; an uppermost aqueous phase solution was transferred into a new RNase free EP tube, added with an equal volume of isopropyl alcohol, mixed well by placing up and down, and allowed to stand at room temperature for 10 min.

After centrifugation at 12,000 rpm for 10 min at 4° C., a white RNA precipitate appeared at the bottom of the tube; the supernatant was discarded, 1 mL of 75% ethanol was added into the tube, gently inverted several times, and centrifuged at 12,000 rpm for 5 min at 4° C.

The liquid in the tube was discarded, and the white RNA precipitate was air-dried at room temperature until it became transparent, and dissolved in 40 μL of the DEPC water.

(2) Reverse Transcription of RNA into cDNA;

The total RNA was reverse transcribed using HiScript II QRT Super Mix for qPCR (+gDNA wiper). The reaction system and reaction conditions were as follows: the reaction system included 950 ng of RNA, 4 μL of 4×gDNA wiper Mix, and ddH₂O making up to 16 μL; after mixing well, a resulting system was placed in a 42° C. water bath for 2 min, added with 4 μL of 5×HiScript II qRT Super Mix II, mixed evenly to allow reverse transcription at 50° C. for 15 min and then 85° C. for 5 s. The obtained cDNA was stored in a −40° C. refrigerator for subsequent qPCR detection.

(3) Quantitative Detection of circRNA PDCoV-S;

The expression level of circRNA PDCoV-S in the cDNA obtained in the previous step was detected using the AceQ qPCR SYBR Green Master Mix kit (Vazyme). A detection system included: 10 μL of 2×AceQ qPCR SYBR Green MasterMix, 0.4 μL each of 10 μM upstream and downstream primers, 4 μL of cDNA, and 5.2 μL of ddH₂O.

A qPCR program included: initial denaturation at 95° C. for 5 min, 1 cycle; 40 cycles of denaturation at 95° C. for 10 s, annealing at 60° C. for 30 s, and extension at 95° C. for 15 s;

60° C. for 30 s and 95° C. for 15 s, 1 cycle.

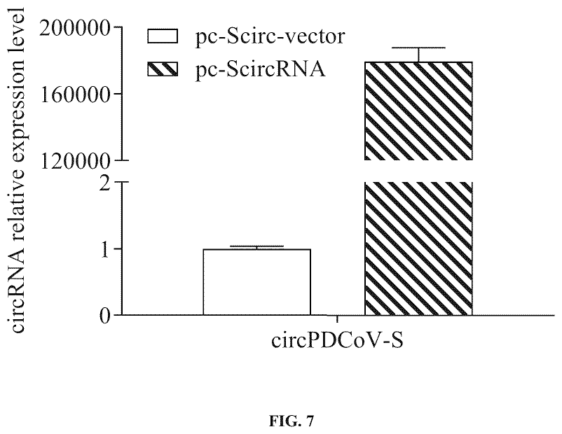

The quantification results showed that the expression level of circRNA in HEK-293 cells transfected with pc-Scirc-PDCoV-S plasmid was 180,000 times higher than that in HEK-293 cells transfected with control plasmid pc-Scirc. The quantitation results of S gene of PDCoV inserted into the circRNA overexpression vector were shown in FIG. 7.

The results of Examples 3 to 6 proved that the circRNA expression frame constructed in the present disclosure could be used to express the circRNA efficiently.

It should be noted that the above are only preferred examples of the present disclosure and are not intended to limit the present disclosure. Although the present disclosure is described in detail with reference to the foregoing examples, it will be apparent to those skilled in the art that modifications can be made to the technical solutions described in the foregoing examples, or some of the technical features can be equivalently replaced. Any modification, equivalent substitution, improvement, etc. within the spirit and principles of the present disclosure shall fall within the scope of protection of the present disclosure.

What is claimed is:

1. A high-efficiency expression vector of a circular ribonucleic acid (circRNA), comprising a nucleotide sequence shown in SEQ ID NO: 1; wherein
a construction method of the high-efficiency expression vector of a circRNA comprises the following steps:
designing amplification primers, conducting PCR amplification using a synthesized circRNA-expressing frame sequence as a template, and adding a homologous arm sequence of 25 bp to two ends of the frame sequence separately, to ligate a resulting entire expression framework sequence to an expression vector pcDNA3.1 (+); and retaining an EcoR I restriction site at a junction between an upstream frame sequence and a downstream frame sequence for inserting a linear nucleotide sequence of a target circRNA; wherein
each of the amplification primers comprises a homologous arm (underlined) and an expression frame complementary sequence, and has nucleotide sequences shown as follows:
Frame-F-5′-TAAACTTAAGCTTGGTACCGAGCTCTGTGAAAACACGGGTTATTCCC-3′ (SEQ ID NO: 2),
Frame-R-5′-AGCGGGTTTAAACGGGCCCTCTAGATGAAAACACGGGTTATTC-3′ (SEQ ID NO: 3).

2. The high-efficiency expression vector of a circRNA according to claim 1, comprising the upstream frame sequence, the downstream frame sequence, and the restriction site for inserting an exogenous circRNA linear sequence.

3. The high-efficiency expression vector of a circRNA according to claim 2, wherein the restriction site for inserting the exogenous circRNA linear sequence is EcoR I.

4. The high-efficiency expression vector of a circRNA according to claim 2, wherein a sequence of 1 bp to 92 bp is the upstream frame sequence, a sequence of 93 bp to 98 bp is the restriction site EcoR I, and a sequence of 99 bp to 173 bp is the downstream frame sequence in the circRNA-expressing frame sequence.

5. The high-efficiency expression vector of a circRNA according to claim 1, wherein an amplification system of the PCR amplification using the frame sequence as the template in the construction method is 50 μL, comprising: 25 μL of a 2× Max buffer, 1 μL of dNTP, 2 μL of each of upstream and downstream primers (10 mM), 1 μL of a synthesized frame sequence DNA template (100 ng), 1 μL of a Phanta® Max Super-Fidelity DNA Polymerase, and sterile water as the balance to 50 μL; and
a reaction program comprises: initial denaturation at 95° C. for 5 min; 30 cycles of denaturation at 95° C. for 30 s, annealing at 56° C. for 30 s, and extension at 72° C. for 30 s; extension at 72° C. for 5 min; and storage at 4° C.

6. A method for efficiently expressing an exogenous target gene/a circRNA, comprising: cleaving a vector at an EcoR I site of a circRNA overexpression frame using an EcoR I enzyme, and then directly recombining a nucleotide sequence of a target gene into the circRNA expression frame to allow expression; wherein the circRNA expression frame has a nucleotide sequence shown in SEQ ID NO: 1.
